(19) United States
(12) Patent Application Publication
(10) Pub. No.: US 2009/0294313 A1
(43) Pub. Date: Dec. 3, 2009

(54) SINGLE-USE MEDICAL EQUIPMENT PACKAGE COVER

(76) Inventors: Jack Pacey, Vancouver (CA); Mitchell Visser, Burnaby (CA); Reza Ahmadian Yazdi, Richmond (CA)

Correspondence Address: BLACK LOWE & GRAHAM, PLLC, 701 FIFTH AVENUE, SUITE 4800, SEATTLE, WA, 98104, US

(21) Appl. No.: 12/368,298

(22) Filed: Feb. 9, 2009

Related U.S. Application Data
(60) Provisional application No. 61/027,377, filed on Feb. 8, 2008.
(60) Provisional application No. 61/029,268, filed on Feb. 15, 2008.

(51) Int. Cl. A61B 19/02

(52) U.S. Cl. 206/370; 206/363

(57) ABSTRACT

A single-use medical equipment package cover comprising a solid preformed shell to cover the functional part of a medical instrument or surgical device and a trailing soft plastic cover or plastic sheath for the rest of the surface of the medical instrument or surgical device. The medical equipment package cover is applied in a folded or stowed configuration and is then unfolded to a deployed configuration. The trailing plastic cover may be transparent and removably affixable to a lens surface or a monitor surface. Unfolding of the removably affixed sheath imparts an interpersonal barrier to block aerial or sputum transfer between patient and medical personnel operating medical equipment being deployed on and within the patient.

### CROSS REFERENCES To RELATED APPLICATIONS

This application claims priority to and incorporates by reference in their entirety U.S. Provisional Patent Application Nos. 61/027,377 filed Feb. 8, 2008 and 61/029,268 filed Feb. 15, 2008.

### COPYRIGHT NOTICE

This disclosure is protected under United States and International Copyright Laws. © 2009 Verathon Inc. All Rights Reserved. A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to the fields of anesthesiology and pre-hospital and emergency airway management.

### BACKGROUND OF THE INVENTION

Some general background relating to, and illustrative devices and systems that can benefit from or work with this invention is described in our co-pending applications U.S. Ser. No. 11/925,868 filed Oct. 27, 2007; U.S. patent application Ser. No. 11/285,743 filed Nov. 21, 2005; and Issued U.S. Pat. Nos. 6,543,447 and 6,655,377; all of which are incorporated by reference herein. The present invention and system includes several additional improved features, enhancements and/or properties, including, without limitation, which are more fully described in the Detailed Description below.

Endotracheal intubation provides the current preferred method for control of the airway for mechanical ventilation. The laryngoscope has been used to place these breathing tubes into the airway, but many distal portions or arms of the laryngoscope can become susceptible to microbial contamination. The scope portion of the laryngoscope needs a high level disinfection to be used in subsequent patients, leading to delay and reduced cost effectiveness. Other surgical related instruments need to maintain sterility in order to perform biopsies and excisions where cleaning issues create significant hardship for the medical staff providers, especially under field use scenarios that lack the structure of a suitably equipped hospital operating room.

### BRIEF DESCRIPTION OF THE DRAWINGS

Preferred and alternative examples of the present invention are described in detail below with reference to the following drawings:

FIG. 1 schematically depicts a folded configuration of a single-use medical equipment package cover;

FIG. 2 schematically depicts the deployed configuration of the single-use medical equipment package cover;

FIG. 3 depicts an alternate embodiment of the deployed single-use medical equipment package cover; and

FIG. 4 depicts another embodiment of the deployed single-use medical equipment package cover.

### DETAILED DESCRIPTION OF THE PARTICULAR EMBODIMENTS

The form of the laryngoscope with which certain embodiments of the invention may be used advantageously may be similar or identical to one or more of the following or variations thereof: GlideScope™ Video Laryngoscope, GlideScope™ Cobalt, GlideScope™ Ranger, Macintosh, Improved Vision Macintosh, Fink, Oxiport Macintosh, Tull Macintosh, Bizzari/Guiffrida, Paed Heine, Soper, Seward, Robertshaw, Saling, Miller, Miller modified, Oxiport Miller, Tull Miller, Wisconsin, Wisconsin/Foregger, Wis-Foregger, Wis-Hipple, Schapira, Snow, Bennet, Alberts, Michaels, Heine, Flagg, Guedel, Bennet, Eversole, Whitehead, Seward, Phillips, Racz-Allen, Magill, Oxford Infant, English Macintosh, Bainton, Double Angle, Blechman, Belscope, Bullard, Upsher, Cranwall, Anderson-Magill, Jachson-Wisconsin, MacCoy, Lowell, Anterior Commissure, Roberts, Ough, Augustine, Franicevic, Wu-Scope, Spector, McGrath series video laryngoscope, Lee or Flam laryngoscope and/or laryngoscope spatula or a modified version of the above-mentioned laryngoscopes and/or spatulas.

A single-use medical equipment package cover that includes a device with a hard shell component and a connected or connectible flexible sheath component, both components sterilizable and configured to maintain sterility and/or hinder the microbial contamination of an instrument, device, or other item to be packaged. The hard shell component is suitably transparent to allow substantially distortion free viewing through the hard shell and configured to reduced glare from light sources, including adjacent lighting. The transparent hard shell component may also be heated to reduce fogging that would otherwise obscure viewing of the packaged equipment. The flexible sheath component envelops the hard shell component to wrap the instrument, device, or container and thereby establish an interpersonal barrier between a user and the instrument, device, or container.

The advent of more intense use of medical equipment related devices in intimate contact with patients has led to the need to provide protective cover to the medical equipment to avoid costly cleaning cycles. The single-use medical equipment package cover meets the requirement in a more economical manner to provide interpersonal barriers for effective prevention of cross infection from patient to patient or from ambient infectious sources to patients. The use of sterile covers, preferably, requires that the cover be an essentially complete barrier, and that it may be placed and removed without compromising the effectiveness of the system. The methods for placing and removing the devices are designed so that the process is predictable and simple. An embodiment of the single-use medical equipment package may be used to cover surgical devices such as, for example, endoscopes and surgical retractor systems and other tools.

An embodiment of the single-use medical equipment package cover includes a solid preformed shell to cover the functional part of a medical instrument or surgical device and a trailing soft plastic cover or plastic sheath for the rest of the surface of the medical instrument or surgical device. The medical equipment package cover is applied in a folded or stowed configuration and is then unfolded to a deployed configuration. The trailing plastic cover may be transparent and removably affixable to a lens surface or a monitor surface. Unfolding of the removably affixed sheath imparts an interpersonal barrier to block aerial or sputum transfer between patient and medical personnel operating medical equipment being deployed on or within the patient. The single-use medical equipment package cover may be sterilizable by autoclave or other sterilizing processes.

Other embodiments of the single-use medical equipment package provide for a sterile packaging device that establishes an interpersonal barrier between a user and an instrument operable by the user and/or operable by other users. The sterile packaging device includes a hard shell that is deployable over a portion of an instrument and a pliable sheet. The pliable sheet is connected to or connectable with the hard shell, and is deployable over a remaining portion of the instrument, such that the soft shell is pivotable around the hard shell to wrap the instrument to establish the interpersonal barrier.

The single-use medical equipment package also includes embodiments wherein the hard shell may be in the form of a pre-formed shell to cover the functional region of the instrument, such as a lens, that does not substantially disturb or alter the optic properties of the lens as viewed by the user. The pre-formed shell may be suitably transparent to permit substantially distortion free viewing through the lens, or medical device imaging screen that permits a video user to observe the screen display in a manner that avoids glare from nearby lighting. The remaining portion of the lens-based instrument or screen display device may be covered by the sterile trailing portion of a flexible plastic cover that need not have the same optical transparency qualities of the pre-formed shell.

The medical equipment package cover can be applied in a folded or stowed configuration and then unfolded to a deployed configuration. The trailing plastic cover may be transparent and removably affixable to a lens surface or a monitor surface. Unfolding of the removably affixed sheath imparts an interpersonal barrier to block aerial or sputum transfer between patient and medical personnel operating medical equipment being deployed on and within the patient.

The economy and utility of the device is further enhanced by inverting the plastic so that during sterilization the sealed soft plastic extension acts as the packaging of the hard portion. The single-use equipment cover may have added utility to wrap or enclose scope mounted monitors and also provide channels for instruments, liquids, or gasses deployable therefrom. Those portions of the device that do not require high-quality optical visibility or clarity may be carried out using a simple soft plastic sleeve. Other embodiments will be configured to enable the plastic to be intimately applied flat on or adjacent the lens area to achieve preferred optics and to avoid glare from lighting associated with the video viewer. The hard shell/soft shell packaging can have a hard shell or soft shell extension that covers a video monitor mounted on an instrument such as a laryngoscope.

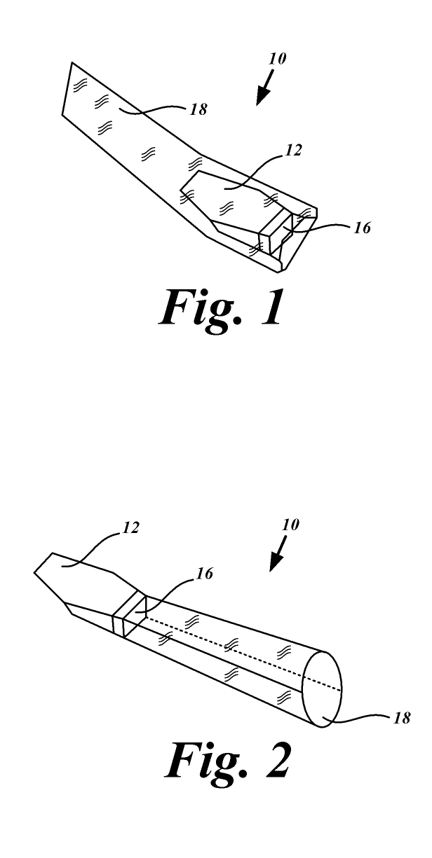

FIGS. 1-4 below illustrate sterile packaging according to an embodiment of medical or surgical equipment common to clinics, hospital, field and/or emergency uses. For example, sterile packaging may include accessory channels for carbon dioxide and oxygen supply equipment, or for passage of surgical instrument equipment, including, for example, flexible suction devices, biopsy forceps, graspers, cutters, and/or holders.

FIG. 1 schematically depicts a folded configuration of a single-use medical equipment package cover. Medical equipment package cover 10 includes a hard shell 12, a scope cover section 16, and a flexible plastic sheath 18.

FIG. 2 schematically depicts the deployed configuration of the single-use medical equipment package cover 10.

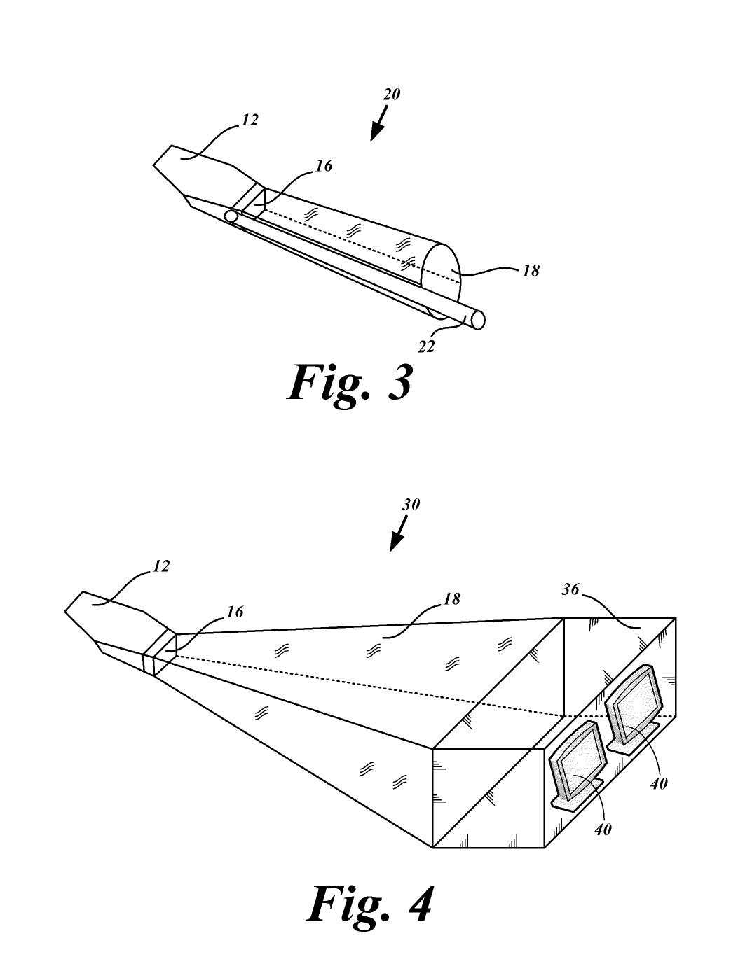

FIG. 3 depicts an alternate embodiment of the deployed single-use medical equipment package cover. Alternate medical equipment package cover 20 includes a channel 22 to convey air lines, electrical lines, or optical lines from the medical equipment into an orifice of a patient (not shown). The channel 22 is pivotable about a point of connection to the hard shell 12 near the scope cover section 16. The channel 22 serves as an accessory channel to convey connectable access to the instrument while maintaining the interpersonal barrier.

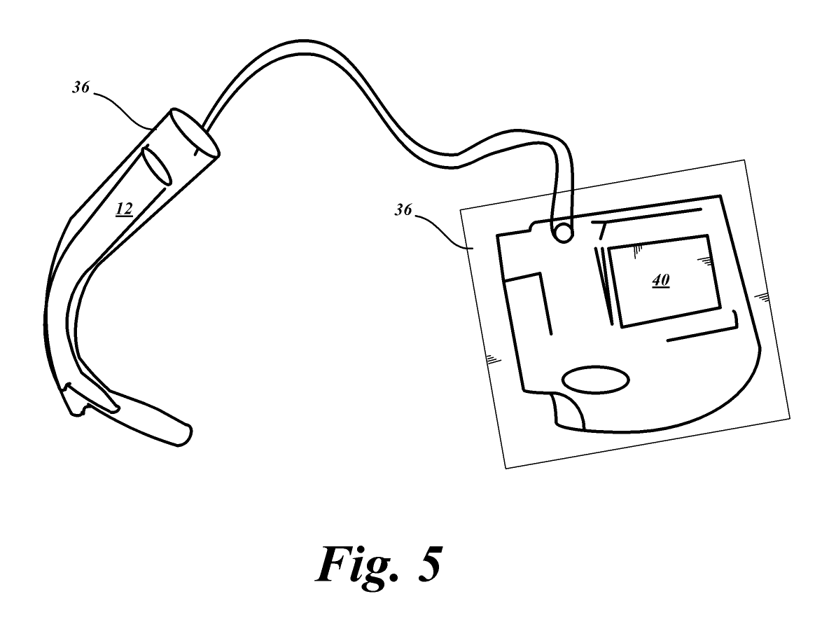

FIG. 4 depicts another embodiment of the deployed single-use medical equipment package cover. Alternate medical equipment package cover 30 includes a second equipment hard shell 36 sealably engageable with the plastic sheath 18. The second hard shell 36 may be configured to cover extensions of nearby scope-mounted monitors.

The use of a partial hard shell—partial soft flexible shell can advantageously provide for design opportunities that are not available with known hard covers. The current video-laryngoscope designs utilize a rigid laryngoscope with a direct view or an optical advantage such as a video camera placed at a point of angulation so the pharyngeal viewpoint may be used to achieve improved access. These video laryngoscopes must be treated with sterilization, high level disinfection, or covering with a sterile single use cover.

Because of the prevalence of Methicillin-Resistant Staphylococcus aureus (MRSA) and other current and future contagion, it is often necessary to provide comprehensive cover for a medical instrument, its associated wiring, its monitor, and any other associated parts. This is achieved by use of a composite medical equipment package cover with a hard shell component and a flexible sheath component.

The hard portion of the shell mechanically locks into position onto the covered medical device so that the operable portion of the device (e.g., the lens of a laryngoscope) is firmly and closely covered by the hard shell, thereby allowing full functioning of the device. The hard shell is secured to the soft sheath extension that covers the remainder of the medical instrument and/or associated wiring, monitor, or other associated parts, thereby giving total protection from contamination to or from the patient. The medical instrument can be placed in the cover and removed from the cover without compromise of the anti-infection protection.

The addition of a firm flexible side channel will allow transit of instruments, fluids, or gas into the working space of the device. The ability to make the flexible sheath cover into the sterile cover of the device is optionally advantageous because it dramatically reduces the bulk of the single use product, it is environmentally friendly, and it is less expensive to produce, ship, store and dispose of after use. The plastics may be recyclable as well.

Where a screen is located on the medical instrument, as in some types of video laryngoscopy, the cover can provide an optical cover for the screen as well. This optical cover may be a hard shell component or a soft shell component as desired. This could be adapted to the video laryngoscopes now in use or new specially-designed scopes.

The hard shell can have a longitudinal channel with an open end and a closed end. The shell can function as a window for viewing carried out by a viewer or camera on a covered device. The shell can be of a close form fit to the video or other device so that the shell of the device is tightly applied to the lens area of the mechanical instrument and has the means to snap in place and hold its position to ensure bi-directional light transmission through the covered medical device, so as to be functionally similar visually to an uncovered medical device. The hard shell can be heated by a heating system carried by the covered instrument.

The shell may include a lifter component that extends the length of the covered device such that multiple sized blades may be created for a single size covered device. The shell and flexible cover may include one or more extra channel for transmission of O₂ gas for improved oxygen delivery in airway work, CO₂ passage for smoke clearing and/or for maintenance of pneumoperitoneum in surgical applications, and/or instrument passage in endoscopic or surgical applications. The relatively inflexible transparent shell may further include a plastic or malleable sheath attached at the open end that extends proximally to cover the entire device and also the wires and/or tubes, if any. The shell may further have the configuration of a malleable sheath that can be inverted over the distal blade cap and sealed to provide sterile packaging solution that is simple and reliable. Other particular embodiments provide for the shell to be comprised of a lubricating material or medicated material within the sealed inverted pouch. The combined hard shell/soft shell embodiments described above may further include a hard or soft shell extension on the proximal portion to cover monitors mounted on laryngoscope or other video instruments to provide sterile cover for the entire instrument.

In one embodiment, the device cover is inside out during storage and transport, such that all the surfaces which might come into contact with the patient or outside environment during use are only in contact with other such surfaces until the device cover is used. In this embodiment, the sterile surfaces which will come into contact with the patient maintain their sterility before use by being only in contact with one another. The operable end of a medical device is inserted into the inside out configuration of the device cover just prior to deployment, and the device cover can then be inverted over the medical device. In this embodiment, the medical device is covered with a disposable sterile cover just prior to use in or on a patient, and that cover is easy to maintain in a sterile configuration due to the storage configuration.

It is advantageous with some medical devices (such as video and fiber optic laryngoscopes, endoscopes, surgical retractors, and the like) that any protective cover mate closely with the operable end of the medical device so that the function of the device is not impeded. For example, laryngoscopes typically include a light source near the operable end which provides illumination of the area of interest to the operator. Video and fiber optic laryngoscopes also include means to transmit images back to the operator, whether through a fiberoptic bundle or through electronic means. A sterile device cover should not impede the image transmission mechanism on such a medical device. In one embodiment of the present invention, the sterile device cover is formed by the combination of a hard shelled portion that is designed to mate closely to the operating end of the medical device and a soft flexible portion that covers the remainder of the device. This allows the sterile device cover to be stored and transported before use in an inside out configuration that helps ensure the sterility of the device before and during its being mounted on the medical device. It also allows the device cover to be firmly and closely mated to the operating end of the medical device while also extending to cover much more of the remainder of the device than the current art of hard shelled only device covers allows.

In a preferred embodiment of the current invention, a rigid portion of the device cover is formed to mate firmly and closely with a particular medical device that requires a disposable sterile sheath. The rigid sheath portion is connected to a flexible portion that can cover the remainder of the medical device. The flexible portion may, for example, be formed of a material that stretches to form-fit closely over a device handle, allowing the operator to more easily manipulate the device. In an alternative embodiment, the flexible plastic portion fits tightly over the more distal portions of the medical device and fits loosely over other portions. In one example, the medical device is a video laryngoscope that has a distal tip that fits into a rigid plastic portion. In this example, the laryngoscope medical device is connected via a wire to a screen in which the operator views the target image. The flexible plastic portion of the medical device cover can extend to substantially cover the wire and screen to minimize the possibility that contaminants might reach the screen or wires, thereby minimizing the need to sterilize those delicate electronic components. In another embodiment, the flexible plastic portion of the medical device cover includes a further rigid portion that fits closely and firmly over the screen to help ensure that the operator's view of the screen is not inhibited by the medical device cover.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A sterile packaging device for an instrument operable by a user comprising:
a hard shell component deployable over a portion of the instrument; and
a pliable sheath component connectable with the hard shell and deployable over the remaining portion of the instrument;
wherein the pliable sheath component envelops the hard shell component to wrap the instrument to establish an interpersonal barrier.

2. The sterile packaging device of claim 1, wherein the hard shell component is transparent to allow the user to view the wrapped instrument.

3. The sterile packaging device of claim 1, wherein the hard shell component is configured to reduce glare from light sources.

4. The sterile packaging device of claim 1, wherein the hard shell component is heated.

5. The sterile packaging device of claim 1 includes an accessory channel to convey connectable access to the instrument while maintaining the interpersonal barrier.

6. A sterile packaging device for an instrument operable by a user comprising:
a hard shell deployable over a portion of an instrument; and
a pliable sheet connectable with the hard shell and deployable over the remaining portion of the instrument;
wherein the pliable sheet is pivotable around the hard shell to wrap the instrument to establish an interpersonal barrier.

7. The sterile packaging device of claim 6, wherein the hard shell component is transparent to allow the user to view the wrapped instrument.

8. The sterile packaging device of claim 6, wherein the hard shell component is configured to reduce glare from light sources.

9. The sterile packaging device of claim 6, wherein the hard shell component is heated.

10. The sterile packaging device of claim 6 includes an accessory channel to convey connectable access to the instrument while maintaining the interpersonal barrier.

11. A sterile packaging device for an instrument operable by a user comprising:
a transparent hard shell deployable over a portion of an instrument;
a pliable sheet connectable with the hard shell and deployable over a remaining portion of the instrument; and
an accessory channel,
wherein the pliable sheet is pivotable around the transparent hard shell to wrap the instrument to establish an interpersonal barrier and the accessory channel provides connectable access to the wrapped instrument while maintaining the interpersonal barrier.

12. The sterile packaging device of claim 11, wherein the transparent hard shell functions as a window for viewing by the user via a camera on the covered device.

13. The sterile packaging device of claim 11, wherein the transparent hard shell is heated.

14. The sterile packaging device of claim 11, wherein the accessory channel conveys at least one of a gas, a surgical device, and an electrical wire.
